(19) United States
(12) Patent Application Publication
(10) Pub. No.: US 2012/0329731 A1
(43) Pub. Date: Dec. 27, 2012

(54) THERAPEUTIC METHODS, COMPOSITIONS, AND COMPOUNDS

(76) Inventors: Herbert T. Nagasawa, St. Paul, CA (US); David J.W. Goon, St. Paul, MN (US); Daune L. Crankshaw, St. Paul, MN (US); Robert Vince, St. Paul, MN (US)

(21) Appl. No.: 13/273,102

(22) Filed: Oct. 13, 2011

Related U.S. Application Data
(63) Continuation of application No. 12/306,899, filed on Mar. 3, 2009, now abandoned.
(60) Provisional application No. 60/817,853, filed on Jun. 30, 2006.

(51) Int. Cl. A61K 31/385; A61K 31/426; A61K 31/554; C07C 321/04; A61K 38/06; C07D 339/08; C07D 285/36; A61P 39/02; A61P 39/06; A61K 31/19; C07D 277/06

(52) U.S. Cl. 514/21.9; 514/557; 514/436; 514/365; 514/211.01; 562/581; 548/201; 549/21; 540/544

(57) ABSTRACT

In one embodiment the invention provides a method of combating cyanide poisoning, which comprises administering to a subject a compound capable of releasing 3-mercapto-pyruvate in vivo. In other embodiments the invention also provides pharmaceutical compositions comprising a compound capable of releasing 3-mercaptopyruvate in vivo, as well as novel compounds that are capable of releasing 3-mercaptopyruvate in vivo.

### PRIORITY OF INVENTION

This application is a continuation of U.S. patent application Ser. No. 12/306,899 filed Dec. 29, 2008, which application is a National Stage application under 35 U.S.C. §371 of International Application No. PCT/US2007/072404 having an International Filing Date of Jun. 28, 2007, and claims priority to U.S. Provisional Application No. 60/817,853 that was filed 30 Jun. 2006, which are incorporated herein by reference in their entireties.

The present invention relates to 3-mercaptopyruvate derivatives. More particularly, it relates to the use of 3-mercaptopyruvate derivatives in the treatment of cyanide poisoning, to novel 3-mercaptopyruvate derivatives, to processes for preparing the novel 3-mercaptopyruvate derivatives, to pharmaceutical compositions comprising 3-mercaptopyruvate derivatives and to a kit comprising 3-mercaptopyruvate derivatives for use in the treatment of cyanide poisoning.

Sulfur compounds have been used for many years in the treatment of cyanide poisoning. Generally, the compounds possess a sulfane sulfur, as in thiosulphate (S—SO₃). The compounds are believed to act as substrates for the mitochondrial enzyme, rhodanese (thiosulfate-cyanate sulfurtransferase, E C 2.8.1.1), which transfers cyanide to the sulfane sulfur of thiosulfate, forming thiocyanide, which is less toxic than cyanide and can be eliminated by the body.

A standard antidote used today in the United States for cyanide poisoning is a combination of sodium nitrite and sodium thiosulfate, which is administered intravenously. The sodium nitrite oxidizes hemoglobin to methemoglobin, which binds cyanide more avidly than cytochrome oxidase, and so spares the latter. In Europe, other cyanide antidotes such as hydroxycobolamine (France) and 4-dimethylaminophenol (Germany) are used.

Unfortunately, ionic thiosulfate is poorly transported across cell membranes to reach the mitochondria, and rhodanese is mainly compartmentalised in the mitochondrial matrix of the liver and kidneys, leaving other tissues, such as the heart and nervous system, poorly protected by thiosulfate treatment. Nitrites, thiosulfates and hydroxycobolamine must all be administered intravenously.

Baskin et al., J. Appl. Toxicol. 19, 173-183 (1999), describe the results of evaluating certain sulfur donors as antidotes to acute cyanide poisoning. The authors comment that the sulfur transferase, 3-mercaptopyruvate sulfur transferase, has been identified as another candidate enzyme to detoxify cyanide. However, they note that several investigators have been unable to find an antidotal effect of 3-mercaptopyruvate, and that no better substrates than 3-mercaptopyruvate have been found for this enzyme.

It has now been found that certain derivatives of 3-mercaptopyruvate protect mice against cyanide poisoning, and further that these compounds are active upon oral administration.

Without wishing to be bound by theory, it is possible that the derivatives of 3-mercaptopyruvate release 3-mercaptopyruvate in vivo, (i.e. function as pro-drugs for 3-mercaptopyruvate) and this compound functions as a substrate for the enzyme 3-mercaptopyruvate sulfurtransferase (3-mercaptopyruvate-cyanide sulfurtransferase, E C 2.8.1.2), which converts the cyanide to thiocyanate and pyruvate.

According to one aspect, therefore, the present invention provides a method of combating cyanide poisoning in a subject in need of treatment, which comprises administering to said subject an effective amount of a compound capable of releasing 3-mercaptopyruvate in vivo.

### BRIEF DESCRIPTION OF THE FIGURES

FIG. 1 depicts the effect of cyanide dose on (a) time required for recovery of neuromuscular coordination, and (b) survival. The left y-axis shows the time in min required for the recovery of the righting reflex, while the right y-axis indicates percent survival. The optimum dose for antidote screening was 4.8 mg CN/kg body weight, with a resultant 68 minutes average time required for the recovery of the righting reflex (shown at the arrow). The survival rate at this optimum dose was 94 percent. The estimated LD₅₀ is at a cyanide dose of 6.1 mg/kg (shown as the dark circle). Values reflect means±SE, n=average 8/group, except control, i.e., 0 cyanide n=20.

FIG. 2 depicts the x-ray crystal structure of (E)-O,S-diacetyl-2-hydroxyl-3-mercapto-2-propenoic acid (1).

FIG. 3 depicts the x-ray crystal structure of 2,5-dihydroxy-2,5-dicarbethoxy-1,4-dithiane (2). The structure is the one suggested. The molecule lies on an inversion center so one-half of the atoms are unique and Z′=½. The hydroxyl proton forms a hydrogen bond with the ester C═O oxygen acceptor on a symmetry related molecule to form dimers. Data collection and structure solution were conducted by Victor G. Young at the X-Ray Crystallographic Laboratory, S146 Kolthoff Hall, Department of Chemistry, University of Minnesota. All calculations were performed using Pentium computers using the current SHELXTL suite of programs.

FIG. 4 depicts the x-ray crystal structure of disodium 2,5-dihydroxy-1,4-dithiane-2,5-dicarboxylic acid tetrahydrate. The structure is the one suggested. The molecule is centered on an inversion center. The sample is held by extensive hydrogen bonding. Data collection and structure solution ware conducted by Benjamin E. Kucera and Victor G. Young, jr., at the X-Ray Crystallographic Laboratory, S146 Kolthoff Hall, Department of Chemistry, University of Minnesota. All calculations were performed using Pentium computers using the current SHELXTL suite of programs.

The subject may be a human or non-human animal, such as a mouse, cat, dog, sheep, goat, cow, pig or horse. In one embodiment of the invention the subject is a human.

The term “effective amount” refers to the dose required to reduce or eliminate one or more of the symptoms of cyanide poisoning.

In general, the dose, or effective amount, will depend upon the weight, age and sex of the subject, the route of administration, and the degree of cyanide poisoning to which the subject has been subjected. The dose will typically be in the range of from 0.1 to 1.5 mmol/kg, such as from 0.145 to 1.45 mmol/kg.

The term combating cyanide poisoning includes prophylactic use as well as treatment of a subject that has already been exposed to cyanide.

In one embodiment of the invention, the compound capable of releasing 3-mercaptopyruvate is administered to the subject as quickly as possible, preferably within five minutes of an exposure to cyanide.

The compound capable of releasing 3-mercaptopyruvate in vivo may be, for example, a compound that can dissociate non-enzymatically to 3-mercaptopyruvate, or that contains one or more bioreversible functional groups that can be removed by enzyme action in vivo to afford 3-mercaptopyruvate.

The compound capable of releasing 3-mercaptopyruvate in vivo may be, for example, a metabolically labile ester or amide of the enol form of 3-mercaptopyruvate, 2,5-dihydroxy-1,4-dithiane-2,5-dicarboxylic acid or 3-mercaptopyruvate; ketone-masked 3-mercaptopyruvate capable of releasing 3-mercaptopyruvate through metabolic or non-enzymatic removal of the ketone mask, or a metabolically labile ester or amide thereof; or a disulfide compound capable of metabolically releasing 3-mercaptopyruvate through reductive cleavage of the sulfur-sulfur bond, or a metabolically labile ester or amide thereof, or a pharmaceutically acceptable salt thereof.

The metabolically labile ester or amide may be any metabolically labile ester or amide formed from a physiologically-tolerable acid, alcohol or amine, for example a (1-6C)alkanoic acid that may bear one, two or three substituents selected from hydroxy, (1-6C)alkoxy, (1-6C)alkanoyloxy, carboxy and (1-6C)alkoxycarbonyl, such as acetic acid or succinic acid; a (1-6C)alkanol that may bear one, two or three substituents selected from hydroxy, (1-6C)alkoxy, (1-6C)alkanoyloxy, amino, (1-6C)alkylamino, di-(1-6C)alkylamino, carboxy and (1-6C)alkoxycarbonyl, such as methanol, ethanol or 2-(N,N-dimethylamino)ethanol, or an amino acid, such as glycine.

The term “amino acid,” includes but is not limited to residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine).

Examples of compounds capable of releasing 3-mercaptopyruvate in vivo are:—

for a metabolically labile ester or amide of the enol form of 3-mercaptopyruvate, a compound of general formula (I)

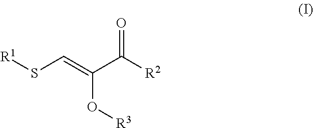

in which R¹ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; R² represents a hydroxyl group, a (1-6C)alkoxy group, NRₐR_{b} (wherein each Rₐ and R_{b} is independently H or (1-6C)alkyl), or a residue of an amino acid; and R³ represents a (1-6C)alkanoyl group that may bear one, two or three substituents selected from hydroxy, (1-6C)alkoxy, (1-6C)alkanoyloxy, carboxy and (1-6C)alkoxycarbonyl, or a pharmaceutically acceptable salt thereof;

for a metabolically labile ester or amide of 2,5-dihydroxy-1,4-dithiane-2,5-dicarboxylic acid, a compound of general formula (II)

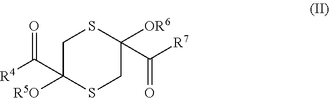

in which each of R⁴ and R⁷ independently represents a hydroxyl group, a (1-6C)alkoxy group, NRₐR_{b} (wherein each Rₐ and R_{b} is independently H or (1-6C)alkyl), or a residue of an amino acid; and each of R⁵ and R⁶ independently represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl; or a pharmaceutically acceptable salt thereof;

for a metabolically labile ester or amide of 3-mercaptopyruvate, a compound of general formula (III)

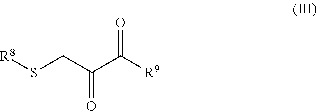

in which R⁸ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl, and R⁹ represents a hydroxyl group, a (1-6C)alkoxy group that may bear one, two or three substituents selected from hydroxy, (1-6C)alkoxy, (1-6C)alkanoyloxy, amino, (1-6C)alkylamino, di-(1-6C)alkylamino, carboxy and (1-6C)alkoxycarbonyl, NRₐR_{b} (wherein each Rₐ and R_{b} is independently H or (1-6C)alkyl), or a residue of an amino acid; or a pharmaceutically acceptable salt thereof;

for ketone-masked 3-mercaptopyruvate, or a metabolically labile ester or amide thereof, a compound of general formula (IV)

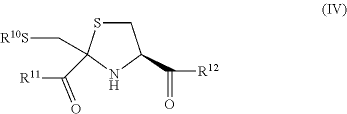

in which R¹⁰ represents a hydrogen atom, a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; one of R¹¹ and R¹² represents a (1-6C)alkoxy group, NRₐR_{b} (wherein each Rₐ and R_{b} is independently H or (1-6C)alkyl), or a residue of an amino acid, and the other of R¹¹ and R¹² represents a hydroxy group, a (1-6C)alkoxy group, NRₐR_{b} (wherein each Rₐ and R_{b} is independently H or (1-6C)alkyl), or a residue of an amino acid, or a pharmaceutically acceptable salt thereof (it will be appreciated that the thiazolidine ring acts as a mask for the ketone group in 3-mercaptopyruvate), and

for a disulfide compound capable of metabolically releasing 3-mercaptopyruvate through cleavage of the sulfur-sulfur bond, or a metabolically labile ester or amide thereof, a compound of general formula (V):

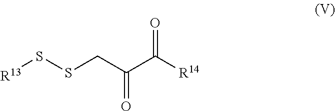

in which:

R¹³ represents R¹⁵C(═O)C(═O)CH₂ or R¹⁶C(═O)CH(NHR¹⁷)CH₂ or a glutathione residue;

R¹⁴ represents a hydroxyl group, a (1-6C)alkoxy group, NRₐR_{b} (wherein each Rₐ and R_{b} is independently H or (1-6C)alkyl), or a residue of an amino acid;

R¹⁵ represents a hydroxyl group or a (1-6C)alkoxy group;

R¹⁶ represents a hydroxyl group or a (1-6C)alkoxy group;

R¹⁷ represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl;

a compound of general formula (VI)

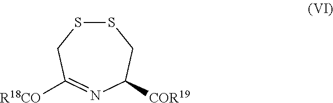

in which:

R¹⁸ and R¹⁹ each independently represents a hydroxyl group, a (1-6C)alkoxyl group, NRₐR_{b} (wherein each Rₐ and R_{b} is independently H or (1-6C)alkyl), or a residue of an amino acid; or

a compound of general formula (VII):

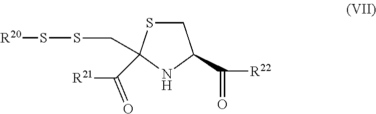

in which:

R²⁰ represents a group of formula HOOCCH(NH₂)CH₂ or

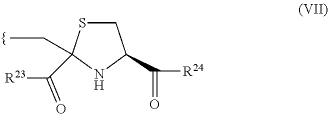

wherein R²¹, R²², R²³ and R²⁴ are each independently selected from a hydroxyl group, a (1-6C)alkoxy group, NRₐR_{b} (wherein each Rₐ and R_{b} is independently H or (1-6C)alkyl), and an amino acid residue.

As used herein, the term “amino acid residue” signifies an amino acid group linked through the amino group of the amino acid to a carbonyl group. An example of an amino acid is glycine.

The term “glutathione residue” signifies a group of formula

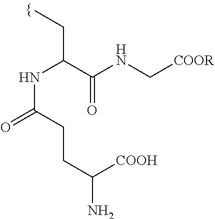

in which R represents a hydrogen atom or a (1-6C)alkyl group. Examples of particular values for a glutathione residue are HO₂CCH₂NHCOCH(NHCOCH₂CH₂CH(NH₂)COOH)CH₂ and EtO₂CCH₂NHCOCH(NHCOCH₂CH₂CH(NH₂)COOH)CH₂.

Unless otherwise indicated, an alkyl group in a (1-6C)alkyl, (1-6C)alkoxy or (1-6C)alkanoyl group may be branched or unbranched, and two branches may join to form a ring, as for example in cyclopropylmethyl.

It will be appreciated that the compounds of formula (I) may exist in the form of geometric isomers. The present invention provides both the (E) and the (Z) isomers. In one embodiment, the compound of formula (I) is in the (E) configuration.

In one embodiment of the invention the compound capable of releasing 3-mercaptopyruvate in vivo is a compound of general formula (I)

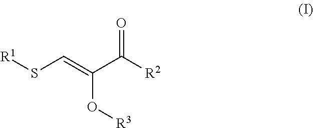

in which R¹ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; R² represents a hydroxyl group, a (1-6C)alkoxy group or a residue of an amino acid; and R³ represents a (1-6C)alkanoyl group that may bear one, two or three substituents selected from hydroxy, (1-6C)alkoxy, (1-6C)alkanoyloxy, carboxy and (1-6C)alkoxycarbonyl, or a pharmaceutically acceptable salt thereof;

for a metabolically labile ester or amide of 2,5-dihydroxy-1,4-dithiane-2,5-dicarboxylic acid, a compound of general formula (II)

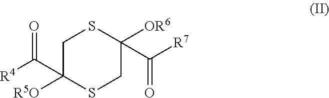

in which each of R⁴ and R⁷ independently represents a hydroxyl group, a (1-6C)alkoxy group or a residue of an amino acid; and each of R⁵ and R⁶ independently represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl; or a pharmaceutically acceptable salt thereof;

for a metabolically labile ester or amide of 3-mercaptopyruvate, a compound of general formula (III)

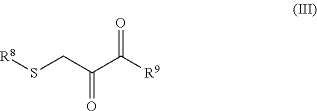

in which R⁸ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl, and R⁹ represents a hydroxyl group, a (1-6C)alkoxy group that may bear one, two or three substituents selected from hydroxy, (1-6C)alkoxy, (1-6C)alkanoyloxy, amino, (1-6C)alkylamino, di-(1-6C)alkylamino, carboxy and (1-6C)alkoxycarbonyl, or a residue of an amino acid; or a pharmaceutically acceptable salt thereof;

for ketone-masked 3-mercaptopyruvate, or a metabolically labile ester or amide thereof, a compound of general formula (IV)

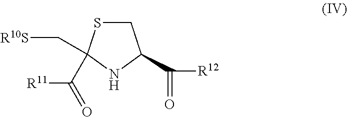

in which R¹⁰ represents a hydrogen atom, a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; one of R¹¹ and R¹² represents a (1-6C)alkoxy group, or a residue of an amino acid, and the other of R¹¹ and R¹² represents a hydroxy group, a (1-6C)alkoxy group, or a residue of an amino acid, or a pharmaceutically acceptable salt thereof (it will be appreciated that the thiazolidine ring acts as a mask for the ketone group in 3-mercaptopyruvate), and

for a disulfide compound capable of metabolically releasing 3-mercaptopyruvate through cleavage of the sulfur-sulfur bond, or a metabolically labile ester or amide thereof, a compound of general formula (V):

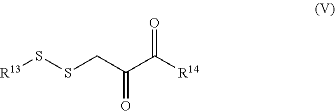

in which:

R¹³ represents R¹⁵C(═O)C(═O)CH₂ or R¹⁶C(═O)CH(NHR¹⁷)CH₂ or a glutathione residue;

R¹⁴ represents a hydroxyl group, a (1-6C)alkoxy group or a residue of an amino acid;

R¹⁵ represents a hydroxyl group or a (1-6C)alkoxy group;

R¹⁶ represents a hydroxyl group or a (1-6C)alkoxy group;

R¹⁷ represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl;

a compound of general formula (VI)

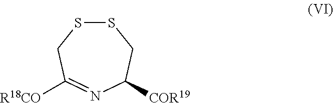

in which:

R¹⁸ and R¹⁹ each independently represents a hydroxyl group, a (1-6C)alkoxyl group or a residue of an amino acid; or

a compound of general formula (VII):

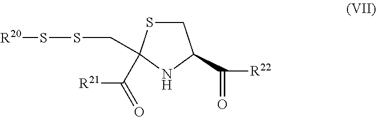

in which:

R²⁰ represents a group of formula HOOCCH(NH₂)CH₂ or

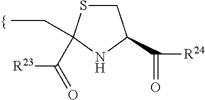

wherein R²¹, R²², R²³ and R²⁴ are each independently selected from a hydroxyl group, a (1-6C)alkoxy group and an amino acid residue.

Examples of particular values are:—

for R¹: ethoxycarbonyl, acetyl or HOOCCH₂CH₂C(═O);
for R²: hydroxy or ethoxy
for R³: acetyl or HOOCCH₂CH₂C(═O);
for R⁴: hydroxy or ethoxy;
for R⁵: hydrogen;
for R⁶: hydrogen;
for R⁷: hydroxy or ethoxy;
for R⁸: ethoxycarbonyl or succinoyl;
for R⁹: hydroxy or OCH₂CH₂N(CH₃)₂;
for R¹⁰: hydrogen, ethoxycarbonyl or succinoyl;
for R¹¹: hydroxy or ethoxy;
for R¹²: hydroxy or HNCH₂COOH;
for R¹³: HOC(═O)C(═O)CH₂, HOC(═O)CH(NH₂)CH₂, HOC(═O)CH(NHAc)CH₂, HO₂CCH₂NHCOCH(NHCOCH₂CH₂CH(NH₂)COOH)CH₂ or EtO₂CCH₂NHCOCH(NHCOCH₂CH₂CH(NH₂)COOH)CH₂;
for R¹⁴: hydroxy, ethoxy or NHCH₂COOH;
for R¹⁵: hydroxy or ethoxy;
for R¹⁶: hydroxy or ethoxy;
for R¹⁷: hydrogen or acetyl;
for R¹⁸: hydroxy;
for R¹⁹: hydroxy or HNCH₂COOH;

### for R²⁰: HOOCCH(NH₂)CH₂ or

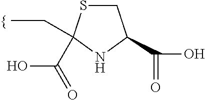

for R²¹: hydroxy;
for R²²: hydroxy;
for R²³: hydroxy; and
for R²⁴: hydroxy.

Examples of particular compounds are:—

for a compound of general formula (I):

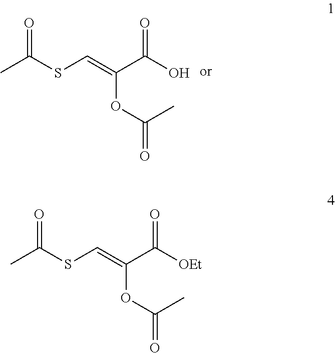

for a compound of general formula (II):

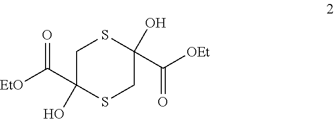

for a compound of general formula (III):

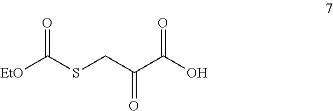

for a compound of general formula (IV):

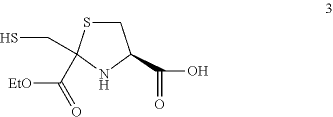

Further examples of compounds capable of releasing 3-mercaptopyruvate are compounds of formulae:

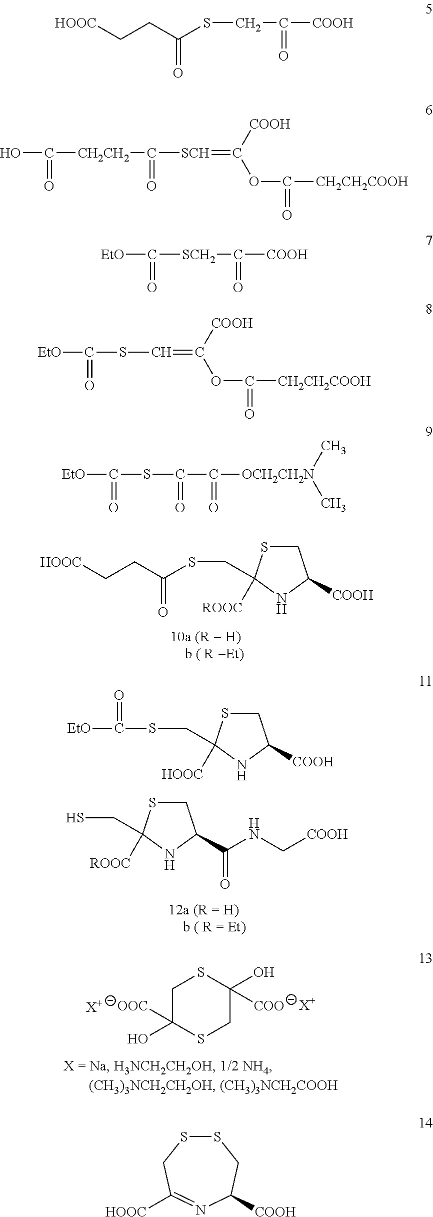

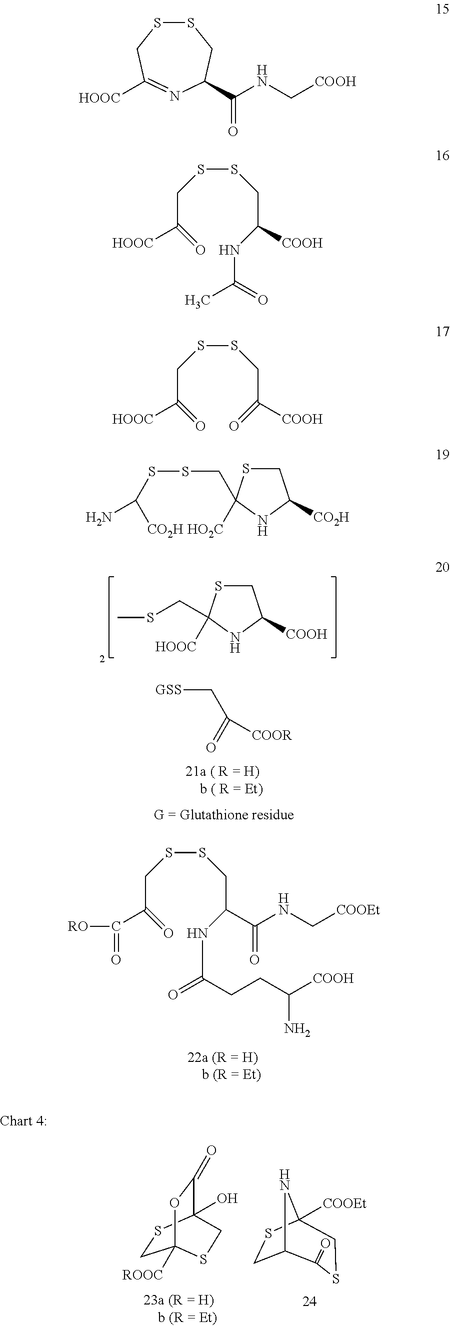

Compound 13, (a compound of formula (II) in which R⁴ and R⁷ each represent hydroxyl groups atoms, and R⁵ and R⁶ each represent hydrogen) is known from Cooper et al., J. Biol. Chem., (1982), 257, 816-826.

Compound 14, (a compound of formula (VI) in which R¹⁸ and R¹⁹ each represents a hydrogen atom) is known, for example from Cavallini D, et al., Advances in Experimental Medicine and Biology (1982), 148, 359-74.

Compound 17, (a compound of formula (V) in which R¹³ represents HOOCCOCH₂ and R¹⁴ represents a hydroxyl group), is also known, for example from Meister et al., Journal of Biological Chemistry (1954), 206, 561-75.

Certain compounds capable of releasing 3-mercaptopyruvate in vivo are believed to be novel. The present invention also provides all such novel compounds.

Compounds capable of releasing 3-mercaptopyruvate in vivo may readily be identified by a person skilled in the art, and may be prepared by conventional synthetic routes.

For example, compounds of general formula (I), (II) and (III) may be prepared by the flow schemes depicted below.

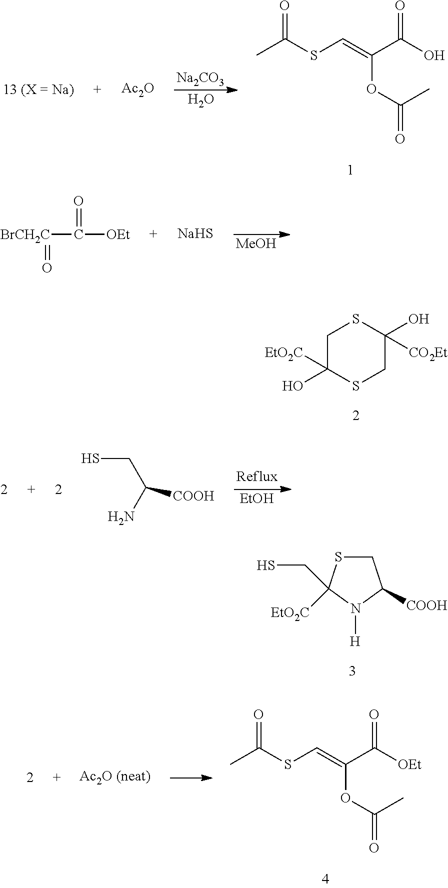

Sodium hydrosulfide was purified by the method of S. Tanabe et al. Sodium 3-mercaptopyruvate, dihydrate may be prepared by the reaction of sodium hydrosulfide and 3-bromopyruvate in methanol according to S. Tanabe et al.
Compound 1 may be prepared by the reaction of acetic anhydride and sodium mercaptopyruvate, dihydrate in aq. sodium carbonate.
Compound 2 may be prepared by the reaction of sodium hydrosulfide with ethyl 3-bromomercaptopyruvate in methanol,
Compound 3 may be prepared by heating under reflux of compound 2, L-cysteine, and absolute ethanol. The cysteine dissolved as 3 was formed.
Compound 4 may be prepared by the reaction of acetic anhydride (neat) with compound 2.
Reference: Tanabe, S.; Ogasawara, Y.; Nawata, M.; Kawanabe, K. Preparation of a sulfurtransferase substrate, sodium 3-mercaptopyruvate, from 3-bromopyruvic acid and sodium hydrosulfide. Chem. Pharm. Bull. 1989, 37, 2843-2845.

Other compounds may be prepared as shown in Schemes 1-2 below:—

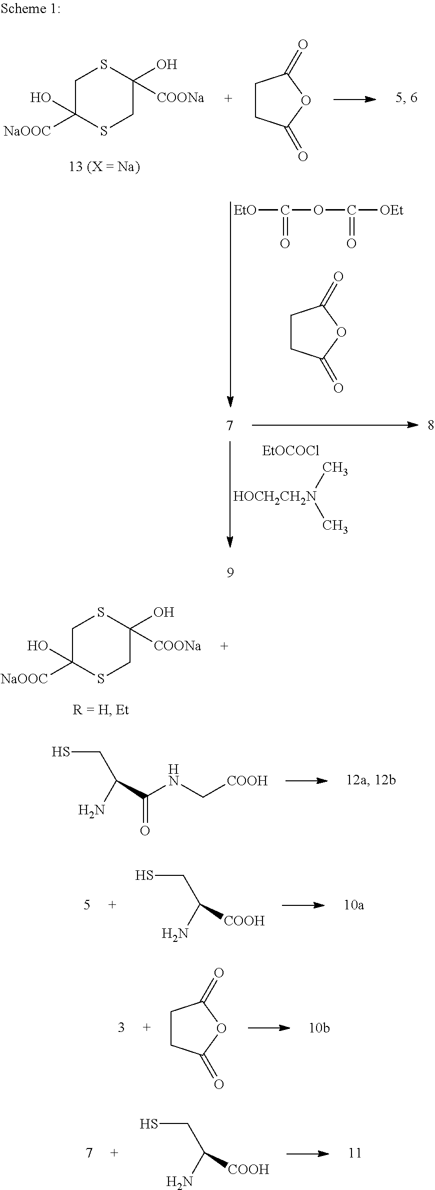

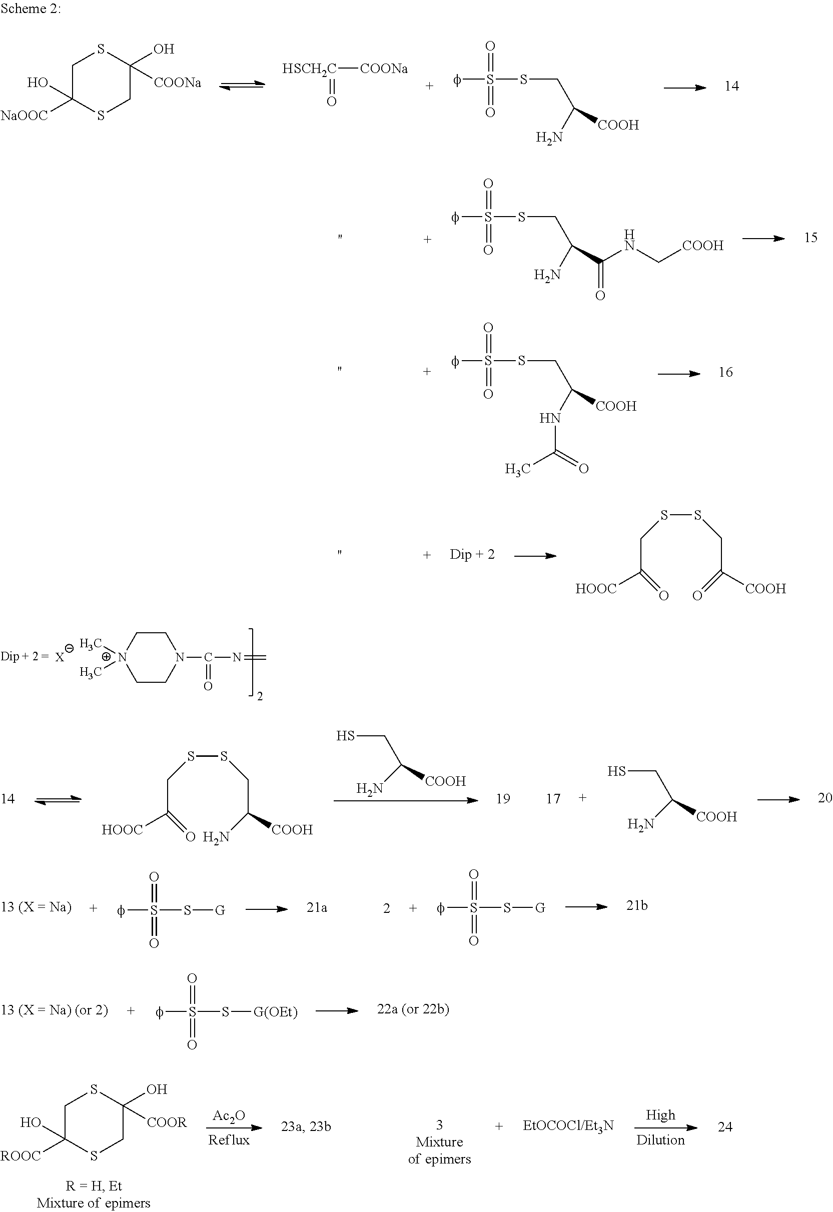

The compounds of the invention may be administered alone, or in combination with one or more other cyanide antidotes, such as a nitrite (typically administered at a dose of about 1.45 mmol/kg), a sulfane sulfur rhodanese substrate, such as thiosulphate (typically administered at a dose of about 6.32 mmol/kg) and hydroxycobolamine.

In one embodiment, the compound is capable of releasing 3-mercaptopyruvate slowly. Such a compound is of particular interest for prophylactic use, i.e. for administration to a subject at risk of exposure to cyanide, and also for co-administration with a compound capable releasing 3-mercaptopyruvate rapidly, for treatment of a subject who might be imminently exposed to cyanide. Examples of such subjects are professionals providing an emergency response or security service, for example firefighters, police officers, medics, and military personnel about to enter a location where cyanide could be released, such as a burning building or damaged chemical plant.

Cyanide poisoning induces multiple toxicities that include not only generation of reactive oxygen species, but also the upregulation of nitric oxide synthase with corresponding increase in NO production and increases in cellular calcium release. Accordingly, in one embodiment of the invention the compounds may be administered in combination with one or more other therapeutic agents that modulate such cellular events (e.g. a compound that reduces oxygen stress or that ameliorates another effect of cyanide poisoning). In one embodiment the compound of the invention is administered in combination with one or more agents that inhibit NO production and/or calcium release. In another embodiment, the compound is administered in combination with a source of an antioxidant, for example a source of glutathione, such as CySSG, the mixed disulfide of L-cysteine and glutathione, which is a bioavailable form of glutathione. An antioxidant, such as glutathione, is useful to protect subjects from neurological deficits associated with cyanide poisoning, and subsequent neurological damage.

In cases where compounds are sufficiently acidic, a salt of a compound of formula I can be useful as an intermediate for isolating, or purifying a compound of formula I. Additionally, administration of a compound of formula I as a pharmaceutically acceptable salt may be appropriate. Pharmaceutically acceptable salts may be obtained using standard procedures well known in the art, for example by reacting a sufficiently acidic or basic compound such as a carboxylic acid with a suitable base, affording a physiologically acceptable cation. Examples of pharmaceutically acceptable base salts are salts formed from organic cations such as, for example, choline and betaine, and other biologically compatible cations. Alkali metal (for example, sodium, potassium or lithium) or alkaline earth metal (for example calcium) salts of carboxylic acids can also be made.

The compounds of the invention may be administered by any convenient route, e.g. into the gastrointestinal tract (e.g. rectally or orally), the nose, lungs, musculature or vasculature or transdermally. The compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

According to another aspect, the present invention provides a pharmaceutical composition, which comprises a 3-mercaptopyruvate derivative as defined herein, together with a pharmaceutically acceptable carrier.

According to another aspect, the present invention provides a kit for use in the treatment of a subject in need of treatment for cyanide poisoning, which comprises a compound capable of releasing 3-mercaptopyruvate in vivo, together with instructions for administration of said compound. In one embodiment, the kit comprises a first compound capable of releasing 3-mercaptopyruvate slowly and a second compound capable of releasing 3-mercaptopyruvate rapidly.

According to another aspect, the present invention provides the use of a compound capable of releasing 3-mercaptopyruvate in vivo in the manufacture of a medicament for the treatment of cyanide poisoning.

The ability of a test compound to combat cyanide poisoning in a subject may be demonstrated in the following in vivo animal model, which is based upon the inverted screen test described by Koplovitz et al., Drug and Chemical Toxicology, 12 (3&4), 221-235 (1989).

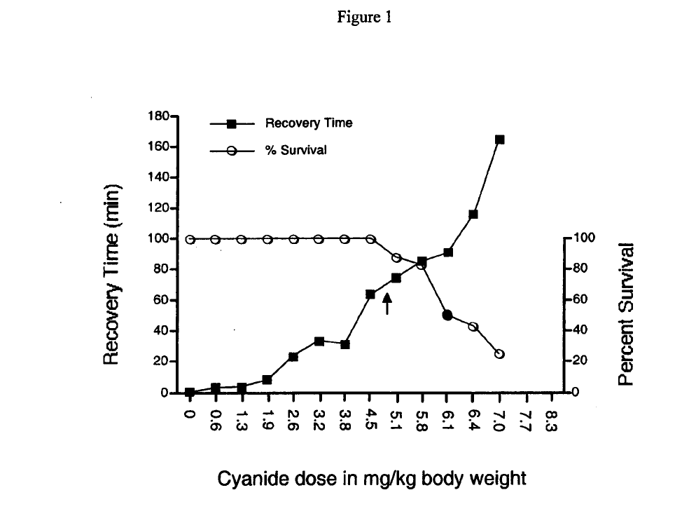

Mice were trained to “right” themselves from an inverted wire mesh screen. Once trained, the mice can rapidly right themselves. A non-lethal, but toxic dose of cyanide (about 4.8 mg/kg) is administered i.p. to the mice such that the recovery based on successful wire mesh righting time, averaged about 68 minutes. Test compounds were then administered at doses described in table legends either intraperitoneally or orally, prior to or after the cyanide dose. The reduction in wire mesh righting time and % survival compared with cyanide alone provide a measure of the efficacy of the test compounds (FIG. 1). In one embodiment the invention also provides the novel assay methods described herein.

Table 1 shows the effect of vehicle and antidote alone on righting reflex. No statistical differences from baseline controls (no treatment) were seen. The sodium salts of cyanide, nitrite and thiosulfate were dissolved in saline. The poor water solubility of the 3-MP prodrugs required the use of DMSO.

TABLE 1
Effect of CN Antidotes and Carrier on Righting Times: i.p. Administration
Screen Righting time (seconds ± SE)

| Treatment | Ave ± SE | Number |
|---|---|---|
| Baseline | 20.7 ± 2.3 | 54 |
| DMSO | 15.8 ± 5.2 | 6 |
| PD 1 | 19.7 ± 4.4 | 6 |
| PD 2 | 21.2 ± 1.2 | 5 |
| PD 3 | 17.5 ± 1.8 | 6 |
| PD 4 | 25.7 ± 4.5 | 6 |
| N/T | 19.0 ± 10.0 | 7 |
| CySSG | 8.8 ± 2.2 | 5 |
| H | 7.1 ± 0.6 | 7 |
| PD 13 (x = Na) | 14.0 ± 2.3 | 8 |

N/T = nitrite and thiosulfate, H = Hydroxocobalamin, CySSG = glutathione prodrug, PD 13 (X = Na) and DMSO = dimethyl sulfoxide. All treatments administered i.p. at the doses (mmol or mg/kg) used in later experiments. Baseline: no treatment. There was no effect of drug or carrier on righting reflex ANOVA (p = 0.2810) as compared to the baseline. Since the analysis showed no difference between treatments, “p values” are not listed in the table.

Tables 2 and 3 summarize the efficacy of Compounds 1-4 as cyanide antidotes, administered i.p., 5 minutes pre- and 5 minutes post-cyanide, respectively, compared to the combination of sodium nitrite and sodium thiosulfate given at the historical, published doses (mouse) of 1.45 and 6.32 mmol/kg, respectively, which are 5× and 22× the antidote dose of 0.29 mmol/kg for the compounds of the invention.

TABLE 2
Effect of 3-MP Prodrugs on Righting Times:
5 min pre-Cyanide; i.p. Administration
Screen Righting Time (minutes ± SE)

| Treatment* | Ave ± SE | N | % S | P Value | P.I. |
|---|---|---|---|---|---|
| CN | 65.9 ± 3.4 | 22 | 76 | | 1.0 |
| CN + PD 1 | 8.7 ± 1.2 | 10 | 100 | <0.0001 | 7.6 |
| CN + PD 2 | 8.7 ± 1.0 | 10 | 100 | <0.0001 | 7.6 |
| CN + PD 3 | 42.5 ± 3.4 | 6 | 100 | <0.002 | 1.6 |
| CN + PD 4 | 10.3 ± 1.3 | 9 | 100 | <0.0001 | 6.4 |
| CN + N/T | 16.4 ± 2.2 | 7 | 100 | <0.0001 | 4.0 |
| CN + H | 12.6 ± 3.3 | 7 | 100 | <0.0001 | 5.2 |

All prodrugs (0.29 mmol/kg) were administered intraperitoneally (i.p.) at 5 min pre-CN (0.10 mmol/kg or 4.8 mg/kg). Values are Means ± SE and statistical analysis was by ANOVA with Scheffe post hoc. Symbols are n = number; % S = percent survivors; P.I. = protective index, i.e., ratio of av. recovery time for CN (+carrier)-treated mice over av. recovery time for PD treated mice. NS = not significant. N/T = Nitrite/thiosulfate (1.45/6.32 mmol/kg). H = Hydroxocobalamin (0.217 mmol/kg = 300 mg/kg). Dimethyl sulfoxide (DMSO) was used as solvent for the hydrophobic CN prodrugs. Dead animals were excluded in the statistical analyses.

TABLE 3
Effect of 3-MP Prodrugs on Righting Times:
5 min post-Cyanide, i.p. Administration
Screen Righting Time (minutes ± SE)

| Treatment* | Ave ± SE | N | % S | P Value | P.I |
|---|---|---|---|---|---|
| CN | 61.3 ± 5.5 | 17 | 94 | | 1.0 |
| CN + PD 1 | 36.9 ± 4.1 | 7 | 100 | NS | 1.7 |
| CN + PD 2 | 15.8 ± 2.3 | 8 | 100 | <0.0001 | 3.9 |
| CN + PD 3 | 42.8 ± 4.0 | 8 | 100 | NS | 1.4 |
| CN + PD 4 | 27.0 ± 3.6 | 8 | 100 | <0.002 | 2.3 |
| CN + N/T | 46.0 ± 5.8 | 8 | 100 | NS | 1.3 |
| CN + H | 44.0 ± 4.0 | 7 | 100 | NS | 1.4 |

*All treatments were administered intraperitoneally (i.p.) at 5 min post-CN.
Other parameters same as Table 2.

It can be seen from the Protective Indices (P.I.) defined here that compounds 1, 2, 3 and 4 administered singly were, generally superior (and a few, far superior) to the combination of nitrite and thiosulfate at much high doses. The total avoidance of the potentially toxic sodium nitrite may also be beneficial here.

The efficacy of compounds 1, 2, 3 and 4 given orally by gavage, 5 minutes pre- and 5 minutes post-CN, in the mouse model. These results are described in Tables 4 and 5, respectively.

Compounds 1, 2, 3 and 4 were found to retain their antidotal activity when administered by the oral route. The convenience and practicality of an orally bioavailable prophylactic agent for cyanide is highly desirable, since the conventional treatments (nitrite and thiosulfate, as well as hydroxycobalamine), must be administered intravenously. The slower action of some compounds is of particular interest for use in a prophylactic anti-cyanide agent.

TABLE 4
Effect of 3-MP Prodrugs on Righting Times:
5 min pre-Cyanide, Oral Administration
Screen Righting Time (minutes ± SE)

| Treatment* | Ave ± SE | n | % S | p Value | P.I. |
|---|---|---|---|---|---|
| CN | 65.2 ± 10.8 | 8 | 73 | | 1.0 |
| CN + PD 1 | 20.4 ± 2.8 | 9 | 100 | <0.001 | 3.2 |
| CN + PD 2 | 45.6 ± 5.4 | 8 | 100 | NS | 1.4 |
| CN + PD 3 | 29.1 ± 4.4 | 7 | 100 | <0.01 | 2.2 |
| CN + PD 4 | 30.4 ± 4.2 | 8 | 100 | <0.0! | 2.1 |

*All prodrugs (0.29 mmol/kg) were administered by gavage 5 mm pre-CN (0.10 mmol/kg or 4.8 mg/kg).
Values are Means ± SE and statistical analysis was by ANOVA with Scheffe post hoc. Symbols are n = number; % S = percent survivors; P.I. = protective index, i.e., ratio of av. recovery time for CN (+carrier)-treated mice over av. recovery time for PD treated mice. NS = not significant. N/T = Nitrite/thiosulfate (1.45/6.32 mmol/kg). H = Hydroxocobalamin (0.217 mmol/kg = 300 mg/kg). Dimethyl sulfoxide (DMSO) was used as solvent for the hydrophobic CN prodrugs. Dead animals were excluded in the statistical analyses.

TABLE 5
Effect of 3-MP Prodrugs on Righting Times:
5 min post-Cyanide, Oral Administration
Screen Righting Time (minutes ± SE)

| Treatment* | Ave ± SE | n | % S | p Value | P.I. |
|---|---|---|---|---|---|
| CN | 61.1 ± 4.4 | 11 | 65 | | 1.0 |
| CN + PD 1 | 39.0 ± 7.4 | 6 | 100 | <0.05 | 1.6 |
| CN + PD 2 | 32.7 ± 3.3 | 6 | 100 | <0.005 | 1.9 |
| CN + PD 3 | 37.5 ± 3.8 | 6 | 100 | <0.03 | 1.6 |
| CN + PD 4 | 17.8 ± 4.1 | 5 | 100 | <0.0001 | 3.4 |

*All prodrugs (0.29 mmol/kg) were administered by gavage 5 min post-CN.
Other parameters same as Table 4.

Additional data demonstrating the oral efficacy of representative compounds, even when administered 30 and 60 minutes before cyanide, are shown in Tables 6-9.

TABLE 6
Effect of 3-MP Prodrugs on Righting Times:
30 min pre-Cyanide, i.p. Administration
Screen Righting Time (minutes ± SE)

| Treatment* | Ave ± SE | N | % S | P Value | P.I |
|---|---|---|---|---|---|
| CN | 69.5 ± 1.2 | 12 | 100 | | 1.0 |
| CN + PD 1 | 36.0 ± 1.6 | 8 | 100 | <0.0001 | 1.9 |
| CN + PD 2 | 35.5 ± 2.7 | 8 | 100 | <0.0001 | 2.0 |
| CN + PD 3 | 54.0 ± 5.2 | 8 | 100 | <0.006 | 1.3 |
| CN + PD 4 | 33.3 ± 2.7 | 8 | 100 | <0.0001 | 2.1 |
| CN + N/T | 12.1 ± 1.2 | 8 | 100 | <0.0001 | 5.7 |
| CN + H | 13.4 ± 1.8 | 8 | 100 | <0.0001 | 5.2 |

*All treatments were administered intraperitoneally (i.p.) at 30 min pre-CN.
Prodrugs 1, 3, and 4 were administered at (0.58 mmol/kg), while other parameters remained the same as Table 2.

TABLE 7
Effect of Prodrugs on Righting Times:
30 min pre-Cyanide, Oral Administration
Screen Righting Time (minutes ± SE)

| Treatment* | Ave ± SE | n | % S | p Value | P.I. |
|---|---|---|---|---|---|
| CN | 69.9 ± 3.4 | 8 | 100 | | 1.0 |
| CN + PD 1 | 24.6 ± 3.9 | 8 | 100 | <0.0001 | 2.8 |
| CN + PD 2 | 15.3 ± 2.5 | 9 | 100 | <0.0001 | 4.6 |
| CN + PD 3 | 33.5 ± 1.3 | 4 | 100 | <0.0001 | 2.1 |
| CN + PD 4 | 13.0 ± 4.1 | 7 | 100 | <0.0001 | 5.4 |
| CN + CySSG | 27.7 ± 2.7 | 12 | 100 | <0.0001 | 2.5 |
| CN + 13 (X = Na) | 17.0 ± 3.3 | 11 | 100 | <0.0001 | 4.0 |

Prodrugs 1, 2, 3, 4, 13 (X = Na), & CySSG were administered by gavage 30 min pre CN at 1.45 mmol/kg. Other parameters same as Table 4.

TABLE 8
Effect of 3-MP Prodrugs on Righting Times:
60 min pre-Cyanide, Oral Administration
Screen Righting Time (minutes ± SE)

| Treatment* | Ave ± SE | n | % S | p Value | P.I. |
|---|---|---|---|---|---|
| CN | 69.8 ± 3.2 | 4 | 100 | | 1.0 |
| CN + PD 3 | 35.3 ± 1.6 | 8 | 100 | <0.0001 | 2.0 |
| CN + PD 4 | 33.9 ± 2.2 | 8 | 100 | <0.0001 | 2.1 |

Prodrugs 3 & 4 were administered by gavage 60 mm pre CN at 1.45 mmol/kg. Other parameters same as Table 4.

TABLE 9
Dose response of Prodrugs on Righting Times:
30 min pre-Cyanide, Oral Administration
Screen Righting Time (minutes ± SE)

| Treatment* | Ave ± SE | n | % S | p Value | P.I. |
|---|---|---|---|---|---|
| CN | 65.8 ± 31.6 | 28 | 100 | | 1.0 |
| CN + PD 2(0.073) | 68.5 ± 3.7 | 6 | 100 | NS | 1.0 |
| CN + PD 2(0.18) | 31.2 ± 2.4 | 6 | 100 | <0.0001 | 2.1 |
| CN + PD 2 (0.36) | 29.3 ± 2.5 | 8 | 100 | <0.0001 | 2.3 |
| CN + PD 2 (0.73) | 30.3 ± 1.2 | 8 | 100 | <0.0001 | 2.2 |
| CN + PD 2 (1.45) | 15.3 ± 2.5 | 9 | 100 | <0.0001 | 4.3 |
| CN + PD 2 (1.74) | 15.7 ± 0.9 | 6 | 100 | <0.0001 | 4.2 |
| CN + CySSG (0.36) | 45.4 ± 3.9 | 5 | 100 | <0.03 | 1.5 |
| CN + CySSG (0.73) | 48.3 ± 1.6 | 9 | 100 | <0.02 | 1.4 |
| CN + CySSG (1.45) | 27.7 ± 2.7 | 12 | 100 | <0.0001 | 2.4 |
| CN + CySSG (2.45) | 19.2 ± 3.4 | 6 | 100 | <0.0001 | 3.4 |
| 13 (X = Na) (0.18) | 26.0 ± 3.9 | 6 | 100 | <0.0001 | 2.5 |
| 13 (X = Na) (0.36) | 31.3 ± 4.1 | 8 | 100 | <0.0001 | 2.1 |
| 13 (X = Na) (0.73) | 14.7 ± 2.5 | 7 | 100 | <0.0001 | 4.5 |
| 13 (X = Na) (1.45) | 17.0 ± 3.3 | 11 | 100 | <0.0001 | 3.9 |

Prodrug #2, CySSG and 13 (X = Na) were administered by gavage 30 min pre CN at mmol/kg noted in parenthesis. Other parameters same as Table 4.

The following non-limiting examples illustrate the invention.

### EXAMPLE 1

### (E)-O,S-Diacetyl-2-hydroxyl-3-mercapto-2-propenoic acid (1)

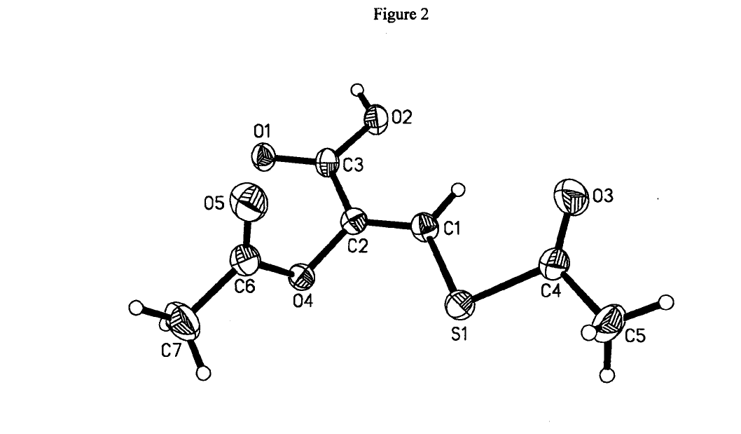

Acetic anhydride (3.06 g, 29.7 mmol) was added dropwise with stirring to a cooled (ice bath) solution of 13 (x=Na) (1.91 g, 5.4 mmol), Na₂CO₃ (3.16 g, 29.8 mmol) and H₂O (50 mL). Stirring and cooling were continued for 1 h, and the pH was adjusted to ˜2 with 6 M HCl, which resulted in the formation of white solids. The mixture was extracted with EtOAc (3×50 mL), and the combined extract was dried over Na₂SO₄. Concentration on a rotary evaporator followed by addition and removal of toluene (2×10 mL, to remove traces of HOAc) and drying the residue in a vacuum desiccator (silica gel) resulted in the isolation of 1.49 g of a white solid. Recrystallization from EtOAc-hexane gave colorless needles (1.00 g, 45.9% yield); mp 140-1; ¹H NMR (CDCl₃) δ 8.05 (s, 1H), 2.49 (s, 3H), 2.28 (s, 3H), ¹³C NMR (CDCl₃) δ 1.87, 167.7, 165.2. 135.2, 125.6. 31.0, 20.2; Anal. (C₇H₈O₅) C, H; x-ray crystal structure (FIG. 2).

TABLE 1
Crystal data and structure refinement for 02294bbb.
Victor G. Young, Jr., X-Ray Crystallographic Laboratory,
Department of Chemistry, University of Minnesota,
207 Pleasant St. S.E. Minneapolis, MN 55455

| Identification code | 02294bbb | |
|---|---|---|
| Empirical formula | C₇ H₈ O₅ S | |
| Formula weight | 204.19 | |
| Temperature | 173(2) K | |
| Wavelength | 0.71073 Å | |
| Crystal system | Monoclinic | |
| Space group | P2₁/c | |
| Unit cell dimensions | a = 5.263(1) Å | α = 90°. |
| | b = 7.029(2) Å | β = 90.685(4)°. |
| | c = 25.909(6) Å | γ = 90°. |

| | |
|---|---|
| Volume | 958.4(4) Å³ |
| Z | 4 |
| Density (calculated) | 1.415 Mg/m³ |
| Absorption coefficient | 0.326 mm⁻¹ |
| F(000) | 424 |
| Crystal habit and color | Colorless, Plate |
| Crystal size | 0.30 × 0.08 × 0.04 mm³ |
| Theta range for data collection | 1.57 to 25.07°. |
| Index ranges | −6 ≦ h ≦ 6, 0 ≦ k ≦ 8, 0 ≦ l ≦ 30 |
| Reflections collected | 9394 |
| Independent reflections | 1735 [R(int) = 0.0490] |
| Observed Reflections | 1370 |
| Completeness to theta = 25.07° | 98.9% |
| Absorption correction | Multi-scan |
| Max. and min. transmission | 0.9871 and 0.8946 |
| Refinement method | Full-matrix least-squares on F² |
| Data/restraints/parameters | 1735/0/122 |
| Goodness-of-fit on F² | 1.026 |
| Final R indices [I > 2sigma(I)] | R1 = 0.0527, wR2 = 0.1516 |
| R indices (all data) | R1 = 0.0674, wR2 = 0.1583 |
| Largest diff. peak and hole | 0.434 and −0.300 e.Å⁻³ |

### EXAMPLE 2

### 2,5-Dihydroxy-2,5-dicarbethoxy-1,4-dithiane (2)

To a cooled (ice/salt bath) solution of ethyl 3-bromopyruvate (11.7 g, 60.0 mmol) and MeOH (40 mL) was added dropwise with magnetic stirring over 1.5 h a cooled (ice bath) solution of sodium hydrosulfide (3.56 g, 63.5 mol) and MeOH (40 mL). White solids formed during the addition, and cooling with stirring was continued for 1 h. After the addition of abs. EtOH (20 mL), the solids were collected, washed with abs. EtOH (20 mL) and dried in a vacuum desiccator to give 5.67 g of product, mp 132-4° C. (uncorr.). Recrystallization from hot MeOH gave products in three crops, 3.10 g, mp 140-3° C., 1.80 g, mp 143-6° C. and 0.50 g, mp 141-3° C.: Total yield, 5.40 g (60.7% yield); Anal. (C₁₀H₁₆O₆S)C, H; ¹H NMR (DMSO-d₆) δ 6.98 (bs, 2H), 4.15 (q, J=7.0 Hz, 4H), 3.62 & 2.85 (AB q, J=14.0 Hz, 4H), 1.20 (t, J=7.0 Hz, 6H); ¹³C NMR (DMSO-d₆) δ 171.3, 77.00, 66.2, 35.6, 14.5; ESI/HRMS (CH₃CN/H₂O) (M+Na−2H) 317.0124 observed, 317.0124 expected, 0 ppm error; (M+Na) 319.0283 observed, 319.0281 expected, 0.91 ppm error; ESI/HRMS (MeOH) (0.5M+Na+MeOH) 203.0348 observed, 203.0349 expected, 0 ppm error.

Compound 2 was also prepared as described in Example 3.

### EXAMPLE 3

### 2,5-Dihydroxy-2,5-dicarbethoxy-1,4-dithiane (2)

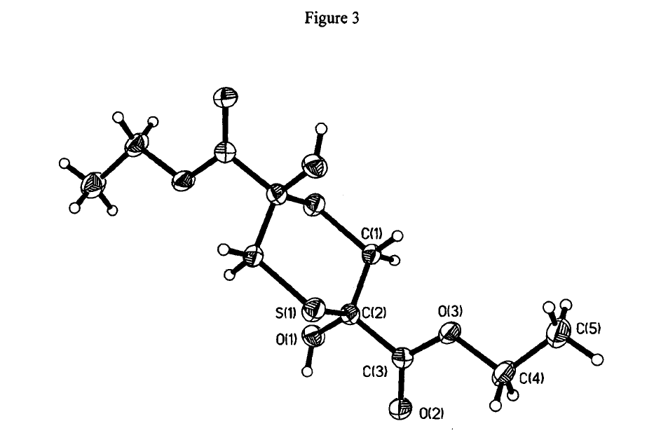

To a cooled (ice/salt bath) solution of ethyl 3-bromopyruvate (12.00 g, 10.8 g corrected for 89.9% purity, 55.4 mmol) and MeOH (30 mL) was added drop wise with magnetic stirring over 45 minutes, a cooled (ice bath) solution of sodium hydrosulfide (3.60, 3.12 g corrected for 86.8% purity, 55.6 mol) and MeOH (20 mL). White solids formed during the addition, and cooling with stirring was continued for 15 minutes. After the addition of abs. EtOH (20 mL), the solids were collected, washed with abs. EtOH (30 mL) and dried in a vacuum desiccator to give 6.14 g. Recrystallization from hot MeOH gave product in two crops, 4.02 g, mp 139-143° and 2.03 g, mp 139-140°; total yield, 6.05 g (73.7%); X-ray structure (FIG. 3).

TABLE 2
Crystal data and structure refinement for compound (2)

| Identification code | 07062a | |
|---|---|---|
| Empirical formula | C10 H16 O6 S2 | |
| Formula weight | 296.35 | |
| Temperature | 173(2) K | |
| Wavelength | 0.71073 Å | |
| Crystal system | Monoclinic | |
| Space group | C2/c | |
| Unit cell dimensions | a = 15.1022(13) Å | α = 90° |
| | b = 6.0189(5) Å | β = 107.467(1)° |
| | c = 15.4114(13) Å | γ = 90° |

| | |
|---|---|
| Volume | 1336.3(2) Å³ |
| Z | 4 |
| Density (calculated) | 1.473 Mg/m³ |
| Absorption coefficient | 0.414 mm⁻¹ |
| F(000) | 624 |
| Crystal color, morphology | Colorless, Plate |
| Crystal size | 0.45 × 0.38 × 0.10 mm³ |
| Theta range for data collection | 2.77 to 25.03° |
| Index ranges | −17 ≦ h ≦ 16, 0 ≦ k ≦ 7, 0 ≦ l ≦ 18 |
| Reflections collected | 6581 |
| Independent reflections | 1181 [R(int) = 0.0225] |
| Observed reflections | 1099 |
| Completeness to theta = 25.03° | 100.0% |
| Absorption correction | Multi-scan |
| Max. and min. transmission | 0.9597 and 0.8355 |
| Refinement method | Full-matrix least-squares on F² |
| Data/restraints/parameters | 1181/0/84 |
| Goodness-of-fit on F² | 1.096 |
| Final R indices (I > 2sigma(I)] | R1 = 0.0267, wR2 = 0.0665 |
| R indices (all data) | R1 = 0.0297, wR2 = 0.0680 |
| Largest diff. peak and hole | 0.270 and −0.170 e. |

### EXAMPLE 4

### Disodium 2,5-dihydroxy-1,4-dithiane-2,5-dicarboxylic acid tetrahydrate 13 (X=Na)

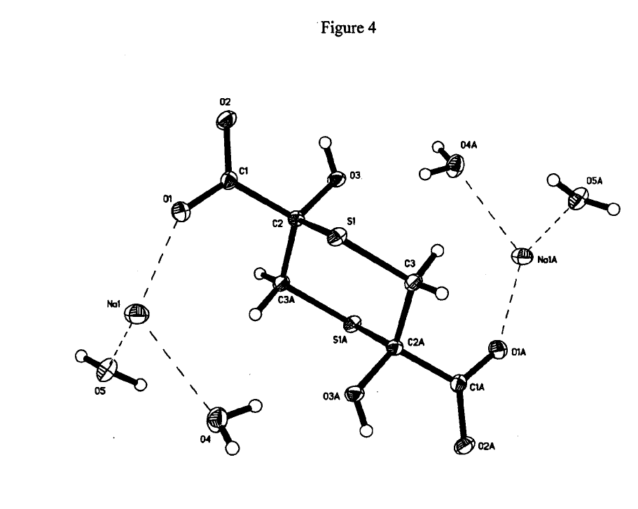

Sodium hydrosulfide was purified by the method of S. Tanabe et al. Purity was assessed by iodometric titration. Purified NaHS was found to be in the range of 69-87%, relative to 65% for the commercial, crude NaHS. Higher purity correlated with how soon the material was assayed after isolation. Thus, it was found that it was best to use the NaHS soon after isolation or to store over sulfuric acid in a vacuum desiccator The titled compound was prepared by the reaction of sodium hydrosulfide and 3-bromopyruvate in methanol according to S. Tanabe et al. However, Tanabe et al. claimed the compound to be a monomer, whereas we determined the compound to be in a dimeric dithiane form (FIG. 4).

TABLE 3
Crystal data and structure refinement for 07061.

| Identification code | 07061 | |
|---|---|---|
| Empirical formula | C₆ H₁₄ Na₂ O₁₀ S₂ | |
| Formula weight | 356.27 | |
| Temperature | 173(2) K | |
| Wavelength | 0.71073 Å | |
| Crystal system | Monoclinic | |
| Space group | P2₁/n | |
| Unit cell dimensions | a = 5.7045(13) Å | α = 90° |
| | b = 6.2569(15) Å | β = 95.754(4)° |
| | c = 18.468(4) Å | γ = 90° |

| | |
|---|---|
| Volume | 655.9(3) Å³ |
| Z | 2 |
| Density (calculated) | 1.804 Mg/m³ |
| Absorption coefficient | 0.518 mm⁻¹ |
| F(000) | 368 |
| Crystal color, morphology | colorless, block |
| Crystal size | 0.45 × 0.45 × 0.38 mm³ |
| Theta range for data collection | 2.22 to 27.52° |
| Index ranges | −7 ≦ h ≦ 7, −8 ≦ k ≦ 8, −23 ≦ |
| | l ≦ 23 |
| Reflections collected | 7281 |
| Independent reflections | 1497 [R(int) = 0.0218] |
| Observed reflections | 1424 |
| Completeness to theta = 27.52° | 99.3% |
| Absorption correction | Multi-scan |
| Max. and min. transmission | 0.8275 and 0.8003 |
| Refinement method | Full-matrix least-squares on F² |
| Data/restraints/parameters | 1497/0/112 |
| Goodness-of-fit on F² | 1.014 |
| Final R indices [I > 2sigma(I)] | R1 = 0.0231, wR2 = 0.0657 |
| R indices (all data) | R1 = 0.0241, wR2 = 0.0665 |
| Largest diff. peak and hole | 0.467 and −0.292 e.Å⁻³ |

### EXAMPLE 5

### 2-Carbethoxy-2-mercaptomethyl-L-thiazolidine-4-carboxylic acid (3)

Compound 2 (2.00 g, 6.75 mmol) was dissolved into 95% EtOH with brief heating on a steam bath. The solution was stirred magnetically and N₂ was bubbled through the solution for 10 min. Finely ground L-cysteine (1.70 g, 14.0 mmol) was added, and the mixture was stirred and heated under reflux under a N₂ atmosphere for 3 h. Most of the L-cysteine appeared to dissolve. The reaction mixture was filtered, and the filtrate was concentrated on a rotary evaporator. The viscous, colorless residue was suspended in 10% NaHCO₃ (40 mL), and the mixture was extracted with EtOAc (3×30 mL). The aqueous layer was acidified with conc. HCl to pH 2, and the resulting mixture was extracted with EtOAc (3×30 mL). The combined extracts were dried over Na₂SO₄ and then concentrated in vacuo. The residue was subjected to the action of a vacuum pump. This produced 3.15 g of a viscous, colorless liquid that partially solidified on standing at room temperature. Recrystallization from EtOAc-hexane was unsuccessful, but removal of the solvent gave a white solid (2.19 g, 64.6% yield): mp 95-98° C.; ¹H NMR (CD₃OD) δ 4.2 (m, 4H), 4.1-2.7 (various m), 1.2 (m, 6H); ¹³C NMR (CD₃OD) δ 170.1, 169.4, 77.7, 64.4, 63.0, 60.5, 37.8, 36.4, 31.3, 30.4, 11.4; Anal. (C₈H₁₃NO₄S)C, H, N; HRMS (M+H) 252.0361 observed, 252.0359 expected, 0.89 ppm error; (M+Na), 274.0186 observed, 274.00179 expected, 2.74 ppm error.^{a} ^{a}The m/e 280.0673 and 302.0490 (M+H and M+Na of the diethyl ester peaks) that are observed in the HRMS of the solvent-free residue of the first EtOAc extract, were absent.

### EXAMPLE 6

### Ethyl-(E)-O,S-Diacetyl-2-hydroxyl-3-mercapto-2-propenate (4)

Compound 2 (0.92 g, 3.1 mmol) and acetic anhydride (5 mL) were heated on a steam cone for 75 min. Compound 2 dissolved within 30 min, and the color changed from yellow to brown during the heating. After cooling to room temperature, 10% Na₂CO₃ (20 mL) was added, and the mixture was stirred for 30 minutes. Extraction with EtOAc (3×20 mL) followed. The combined extracts were dried over Na₂SO₄ and concentrated on a rotary evaporator. Toluene (3×15 mL) was added and then removed in vacuo to give a brown liquid, 1.21 g. The liquid was partially decolorized on a silica gel 60 column (CH₂Cl₂), and after removal of the solvent, a yellow liquid was obtained (0.77 g, 53% yield): ¹H NMR (DMSO-d₆) δ 7.85 (s, 1H), 4.28 (q, J=7.1 Hz, 2H), 2.46 (s, 3H), 2.26 (s, 3H), 1.33 (t, J=7.1 Hz, 3H); ¹³C NMR (DMSO-d₆ δ 188.2, 167.8, 160.1, 136.3, 122.1, 62.0, 309.9, 20.3, 14.2; Anal. (C₉H₁₂O₅S)C, H, S; ESI/HRMS (M+Na) 255.0306 observed, 255.0298 expected, 3.1 ppm error.

### EXAMPLE 7

### O,S-bis-Ethoxycarbonyl-2-hydroxy-3-mercaptopropenoic Acid

Compound 13 (x=Na); (0.61 g, 0.85 mmol) was dissolved in 15 mL of 10% aq. sodium bicarbonate under N₂. To the stirred solution was added ethyl chloroformate (0.95 g, 8.8 mmol) in 20 mL ethyl acetate. The two-phase solution was stirred overnight at room temperature. The aqueous phase (pH 7.3) was acidified to pH ca. 0.8 with 10 mL 1 M aq. HCl and extracted 3× with 20 mL portions of ethyl acetate. The combined ethyl acetate extracts were evaporated to dryness to give 0.61 g of off-white solid. NMR analysis showed the presence of vinyl protons. This crude material was purified by silica gel chromatography. The column was eluted with chloroform and methanol:chloroform (10:90) to give 0.22 g. (24% yield) of 4c as a yellow oil. ¹H NMR (CDCl₃) δ 1.3 (m, 6H, CH₃), 4.2-4.4 (m, 4H, CH₂), 7.77 (br s, 1H, CH), 9.2-9.9 (v br s, OH). ¹³C NMR (CDCl₅) 14.1, 14.2, 65.4, 65.8, 124.8, 137.2, 151.9, 165.0.

### EXAMPLE 8

### Ethyl 4,5,6,7-tetrahydro-4,5-dithioazocine-2-carboxylte

Compound 13 (x=Na); (64 mg, 0.11 mmol) was suspended in ca. 6 mL of H₂O under N₂ and added to a solution of mercaptoethyl amine disulfide monosulfoxide dihydrochloride (61 mg, 0.25 mmol) in 2 mL, H₂O. The mixture was stirred overnight at room temperature at which time the solids had all dissolved. Thin layer chromatography showed that both starting materials were consumed. The solution (pH 2.4) was basified to pH 9.0 by addition of ca. 10 mL of 10% aq. sodium bicarbonate. The solution, which turned light yellow upon basification, was extracted 3× with 10 mL portions of ethyl acetate. The combined extracts were dried with Na₂SO₄ and evaporated to dryness to give 13 mg of yellow-orange oil. NMR analysis showed a vinyl proton suggesting the migration of the double bond following cyclization. ¹H NMR (CDCl₃) δ 1.30 (t, J=7.1 Hz, 3H, CH₃), 2.95 (t, J=5.7 Hz, 4H, CH₂), 4.22 (t, J=7.1 Hz, 2H, CH₂O), 6.02 (s, 1H, CH).

### EXAMPLE 9

### Ethyl O,S-bis-ethoxycarbonyl-2-hydroxy-3-mercapto-propenoate

Compound 13 (x=Na); (0.36 g, 0.61 mmol) was dissolved in 20 mL of dry dioxane under N₂. To the stirred solution was added ethyl chloroformate (0.67 g, 6.2 mmol) in ca. 2 mL dry dioxane, followed by triethyl amine (0.64 g, 6.3 mmol) in about 2 mL dry dioxane over 1 minute. The solution, which turned yellow and formed a precipitate, was stirred overnight at room temperature. The solids were then removed by filtration and rinsed with ethyl acetate. The combined filtrates were evaporated to dryness to give 0.95 g of yellow-orange oil. NMR analysis showed three sets of ethyl peaks and a vinyl proton. This crude material was combined with product from another run (using 0.20 g of dimeric ethyl mercaptopyruvate) and was purified by silica gel chromatography. The column was eluted with ethyl acetate:hexane (7:93 and 10:90) to give 0.53 g. (48% yield, based on the combined runs) of 4b as a pale yellow oil. ¹H NMR (CDCl₃) δ 1.3 (m, 9H, CH₃), 4.2-4.4 (m, 6H, CH₂), 7.70, 7.71 (s, 1H, CH). ¹³C NMR (CDCl₅) 14.10, 14.18, 14.24, 62.0, 65.4, 65.7, 124.3, 136.2, 151.7, 159.7, 164.9. APCI/MS 293 (MH), 247 (MH-EtOH), 219 (100%, MH-EtOH—CO).

### EXAMPLE 10

### Preparation of Compound (12a)

L-Cysteinylglycine (88.6 mg, 0.0498 mmol) and the dimeric sodium mercaptopyruvate (2b, 89.2 g, 0.250 mmol) were dissolved in H₂O (10 mL), and the solution was stirred magnetically while N₂ was bubbled through for 2 minutes. The reaction was then placed under a positive nitrogen atmosphere and stirred magnetically through the weekend at room temp. TLC [silica gel GF, n-PrOH; H₂O (7:3)] was not instructive in revealing the extent of the reaction. Since addition of abs. EtOH (10 mL) did not result in cloudiness, the mixture was concentrated on a rotary evaporator. The resulting white, crystalline solid, mp 100-115° (uncorr), gave a positive Ellman's test for free SH group. MS (neg ion): m/e 279 (M−H⁺).

### EXAMPLE 11

### Preparation of Compound (10b)

To a solution of 3 (1.89 g, 7.52 mmol) in dry THF (100 mL) was added succinic anhydride (0.75 g, 7.49 mmol) followed by TEA (0.76 g, 7.51 mmol). After heating under reflux under N₂ for 3.5 h, TLC [silica gel GF, n-PrOH; H₂O (7:3)] indicated the formation of a product different from 3. Following 6 hours of additional heating under the same conditions, the mixture was concentrated on a rotating evaporator. The resulting yellow oil was dissolved in CHCl₃, and after standing for 1 hour at room temperature, the resulting waxy product was collected by filtration. Since TLC showed the presence of TEA, the crude product was re-dissolved in CHCl₃ (addition of MeOH was necessary to attain solution), and the solution was washed with 1 M H₂SO₄, then water, and dried over Na₂SO₄. TLC showed that TEA was no longer present, but the product and succinic acid had similar Rf values. Removal of the solvent yielded an oil that tested negative with Ellman's reagent. MS (neg. ion): peaks at m/e 553, 499, 250, and 172 with expected molecular ion 351.

### EXAMPLE 12

### Preparation of 13 [X═(CH₃)₃NCH₂CH₂OH]

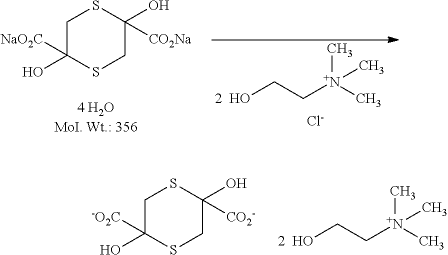

A mixture of Dowex 50 (H⁺; pH 2, 3.3 g) and choline chloride (6.9 g) in water was stirred and then poured into a column. Water in the column was removed by air pressure and the column was washed with water. Choline chloride (5.3 g) in water was loaded to the column, and then the column was washed until choline chloride was no longer detected in the eluent by PMA stain on silica TLC plate. The sodium salt of Compound 13 (x=Na) (66.9 mg) dissolved in water was loaded unto the column and eluted with water. The eluent was passed through the column two additional times. The eluent was concentrated under reduced pressure to give a white solid, 0.10 g, as the dicholine salt. HRMS ESI− calcd for C₁₁H₂₀NO₇S₂: 342.0686, found: 342.0690; ¹H NMR (600 MHz, D₂O) δ: 3.86 (m, 2H), 3.67 (dd, J=28.2 Hz & 2.4 Hz, 1H), 3.00 (s, 9H), 2.69 (dd, J=28.2 Hz & 2.4 Hz, 1H).

1. A method of combating cyanide poisoning in a subject in need of treatment, which comprises administering orally to said subject an effective amount of a compound capable of releasing 3-mercaptopyruvate in vivo.

2. The method as claimed in claim 1, in which the compound is co-administered with another therapeutic agent.

3. The method as claimed in claim 1, in which the compound is co-administered with an antioxidant.

4. The method as claimed in claim 1, in which the compound is co-administered with a source of glutathione.

5. The method as claimed in claim 1, in which the compound is capable of releasing 3-mercaptopyruvate slowly, and is co-administered with a compound capable of releasing 3-mercaptopyruvate rapidly.

6. The method as claimed in claim 1, in which the compound is a metabolically labile ester or amide of the enol form of 3-mercaptopyruvate, a metabolically labile ester or amide of 2,5-dihydroxy-1,4-dithiane-2,5-dicarboxylic acid or a metabolically labile ester or amide of 3-mercaptopyruvate; ketone-masked 3-mercaptopyruvate capable of releasing 3-mercaptopyruvate through metabolic or non-enzymatic removal of the ketone mask, or a metabolically labile ester or amide thereof; or a disulfide compound capable of metabolically releasing 3-mercaptopyruvate through reductive cleavage of the sulfur-sulfur bond, or a metabolically labile ester or amide thereof, or a pharmaceutically acceptable salt thereof.

7. The method as claimed in claim 1, in which the compound is selected from:
(a) a compound of general formula (I) 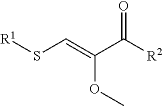
in which R¹ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; R² represents a hydroxyl group, a (1-6C)alkoxy group, NRₐR_{b} (wherein each Rₐ and R_{b} is independently H or (1-6C)alkyl), or a residue of an amino acid; and R³ represents a (1-6C)alkanoyl group that may bear one, two or three substituents selected from hydroxy, (1-6C)alkoxy, (1-6C)alkanoyloxy, carboxy and (1-6C)alkoxycarbonyl, and pharmaceutically acceptable salts thereof;
(b) a compound of general formula (II) 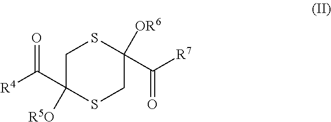
in which each of R⁴ and R⁷ independently represents a hydroxyl group, a (1-6C)alkoxy group, NRₐR_{b} (wherein each Rₐ and R_{b} is independently H or (1-6C)alkyl), or a residue of an amino acid; and each of R⁵ and R⁶ independently represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl; and pharmaceutically acceptable salts thereof;
(c) a compound of general formula (III) 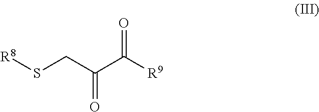
in which R⁸ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl, and R⁹ represents a hydroxyl group, a (1-6C)alkoxy group that may bear one, two or three substituents selected from hydroxy, (1-6C)alkoxy, (1-6C)alkanoyloxy, amino, (1-6C)alkylamino, di-(1-6C)alkylamino, carboxy and (1-6C)alkoxycarbonyl, NRₐR_{b} (wherein each Rₐ and R_{b} is independently H or (1-6C)alkyl), or a residue of an amino acid, and pharmaceutically acceptable salts thereof;
(d) a compound of general formula (IV) 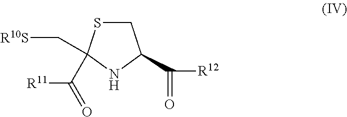
in which R¹⁰ represents a hydrogen atom, a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; one of R¹¹ and R¹² represents a (1-6C)alkoxy group, NRₐR_{b} (wherein each Rₐ and R_{b} is independently H or (1-6C)alkyl), or a residue of an amino acid, and the other of R¹¹ and R¹² represents a hydroxy group, a (1-6C)alkoxy group, NRₐR_{b} (wherein each Rₐ and R_{b} is independently H or (1-6C)alkyl), or a residue of an amino acid, and pharmaceutically acceptable salts thereof;
(e) a compound of general formula (V): 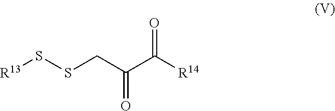
in which:
R¹³ represents R¹⁵C(═O)C(═O)CH₂ or R¹⁶C(═O)CH(NHR¹⁷)CH₂ or a glutathione residue;
R¹⁴ represents a hydroxyl group, a (1-6C)alkoxy group, NRₐR_{b} (wherein each Rₐ and R_{b} is independently H or (1-6C)alkyl), or a reside of an amino acid;
R¹⁵ represents a hydroxyl group or a (1-6C)alkoxy group;
R¹⁶ represents a hydroxyl group or a (1-6C)alkoxy group; and
R¹⁷ represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl; and pharmaceutically acceptable salts thereof;
(f) a compound of general formula (VI): 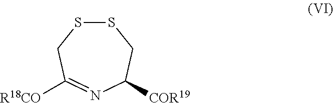
in which:
R¹⁸ and R¹⁹ each independently represents a hydroxyl group, a (1-6C)alkoxyl group, NRₐR_{b} (wherein each Rₐ and R_{b} is independently H or (1-6C)alkyl), or a residue of an amino acid; and pharmaceutically acceptable salts thereof;
(g) a compound of general formula (VII) 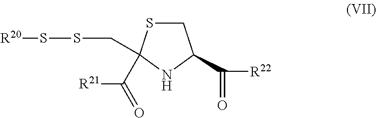
R²⁰ represents a group of formula HOOCCH(NH₂)CH₂ or 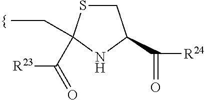
wherein R²¹, R²², R²³ and R²⁴ are each independently selected from a hydroxyl group, a (1-6C)alkoxy group, NRₐR_{b} (wherein each Rₐ and R_{b} is independently H or (1-6C)alkyl), and an amino acid residue; and pharmaceutically acceptable salts thereof; and
(h) a compound of general formula: 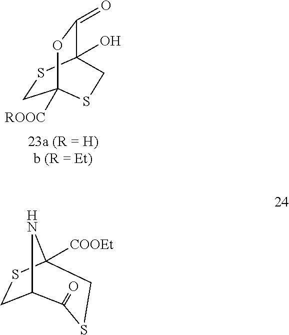
and pharmaceutically acceptable salts thereof.

8. A compound selected from:
(a) a compound of general formula (I) 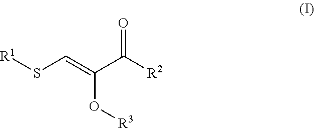
in which R¹ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; R² represents a hydroxyl group, a (1-6C)alkoxy group, NRₐR_{b} (wherein each Rₐ and R_{b} is independently H or (1-6C)alkyl), or a residue of an amino acid; and R³ represents a (1-6C)alkanoyl group that may bear one, two or three substituents selected from hydroxy, (1-6C)alkoxy, (1-6C)alkanoyloxy, carboxy and (1-6C)alkoxycarbonyl;
(b) a compound of general formula (II) 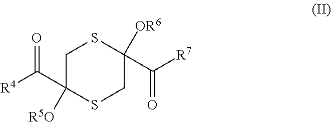
in which each of R⁴ and R⁷ independently represents a hydroxyl group, a (1-6C)alkoxy group, NRₐR_{b} (wherein each Rₐ and R_{b} is independently H or (1-6C)alkyl), or a residue of an amino acid; and each of R⁵ and R⁶ independently represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl; provided that when R⁴ and R⁷ represent hydroxyl groups atoms, R⁵ and R⁶ do not each represent hydrogen;
(c) a compound of general formula (III) 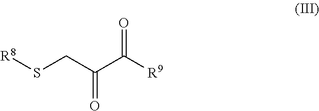
in which R⁸ represents a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl, and R⁹ represents a hydroxyl group, a (1-6C)alkoxy group that may bear one, two or three substituents selected from hydroxy, (1-6C)alkoxy, (1-6C)alkanoyloxy, amino, (1-6C)alkylamino, di-(1-6C)alkylamino, carboxy and (1-6C)alkoxycarbonyl; NRₐR_{b} (wherein each Rₐ and R_{b} is independently H or (1-6C)alkyl), or a residue of an amino acid;
(d) a compound of general formula (IV) 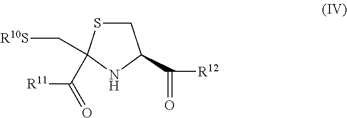
in which R¹⁰ represents a hydrogen atom, a (1-6C)alkoxycarbonyl group or a (1-6C)alkanoyl group that may bear one, two or three substituents selected from hydroxy, (1-6C)alkoxy, (1-6C)alkanoyloxy, carboxy and (1-6C)alkoxycarbonyl; one of R¹¹ and R¹² represents a (1-6C)alkoxy group, NRₐR_{b} (wherein each Rₐ and R_{b} is independently H or (1-6C)alkyl), or a residue of an amino acid, and the other of R¹¹ and R¹² represents a hydroxy group, a (1-6C)alkoxy group, NRₐR_{b} (wherein each Rₐ and R_{b} is independently H or (1-6C)alkyl), or a residue of an amino acid;
(e) a compound of general formula (V): 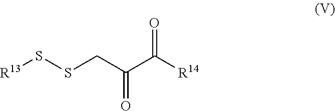
in which:
R¹³ represents R¹⁵C(═O)C(═O)CH₂ or R¹⁶C(═O)CH(NHR¹⁷)CH₂ or a glutathione residue;
R¹⁴ represents a hydroxyl group, a (1-6C)alkoxy group, NRₐR_{b} (wherein each Rₐ and R_{b} is independently H or (1-6C)alkyl), or a residue of an amino acid;
R¹⁵ represents a hydroxyl group or a (1-6C)alkoxy group;
R¹⁶ represents a hydroxyl group or a (1-6C)alkoxy group;
R¹⁷ represents a hydrogen atom or a (1-6C)alkanoyl group that may bear one, two or three substituents selected from hydroxy, (1-6C)alkoxy, (1-6C)alkanoyloxy, carboxy and (1-6C)alkoxycarbonyl, provided that when R¹³ represents HOOCCOCH₂, R¹⁴ does not represent a hydroxyl group;
(f) a compound of general formula (VI): 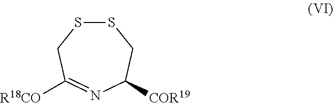
in which:
R¹⁸ and R¹⁹ each independently represents a hydroxyl group, a (1-6C)alkoxyl group, NRₐR_{b} (wherein each Rₐ and R_{b} is independently H or (1-6C)alkyl), or a residue of an amino acid, provided that R¹⁸ and R¹⁹ do not both represent hydrogen atoms;
(g) a compound of general formula (VII): 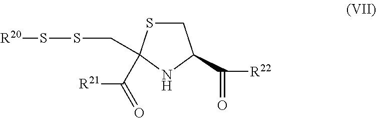
in which R²⁰ represents a group of formula HOOCCH(NH₂)CH₂ or 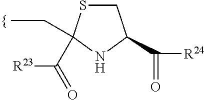
wherein R²¹, R²², R²³ and R²⁴ are each independently selected from a hydroxyl group, a (1-6C)alkoxy group, NRₐR_{b} (wherein each Rₐ and R_{b} is independently H or (1-6C)alkyl), and an amino acid residue; and
(h) a compound of general formula: 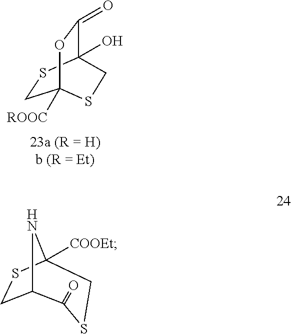
and pharmaceutically acceptable salts thereof.

9. The compound as claimed in claim 8, in which:
R¹ is ethoxycarbonyl, acetyl or HOOCCH₂CH₂C(═O);
R² is hydroxy or ethoxy;
R³ is acetyl or HOOCCH₂CH₂C(═O);
R⁴ is hydroxy or ethoxy;
R⁵ is hydrogen;
R⁶ is hydrogen;
R⁷ is hydroxy or ethoxy;
R⁸ is ethoxycarbonyl or succinoyl;
R⁹ is hydroxy or OCH₂CH₂N(CH₃)₂;
R¹⁰ is hydrogen, ethoxycarbonyl or succinoyl;
R¹¹ is hydroxy or ethoxy;
R¹² is hydroxy or HNCH₂COOH;
R¹³: is HOC(═O)C(═O)CH₂ or HOC(═O)CH(NH₂)CH₂, HOC(═O)CH(NHAc)CH₂, HO₂CCH₂NHCOCH(NHCOCH₂CH₂CH(NH₂)COOH)CH₂ or EtO₂CCH₂NHCOCH(NHCOCH₂CH₂CH(NH₂)COOH)CH₂;
R¹⁴ is hydroxy, ethoxy or NHCH₂COOH;
R¹⁵ is hydroxy or ethoxy;
R¹⁶ is hydroxy or ethoxy;
R¹⁷ is hydrogen or acetyl;
R¹⁸ is hydroxy;
R¹⁹ is hydroxy or HNCH₂COOH;
R²⁰ is HOOCCH(NH₂)CH₂ or 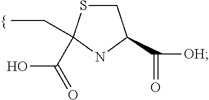
R²¹ is hydroxy;
R²² is hydroxy;
R²³ is hydroxy; and
R²⁴ is hydroxy.

10. The compound as claimed in claim 8, which is selected from:

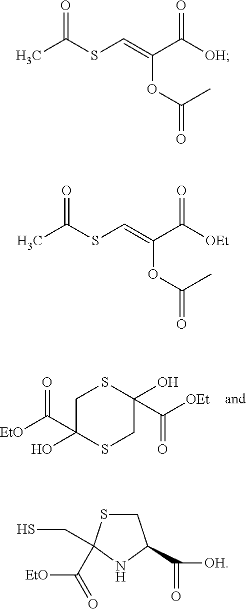

11. The method of claim 1 wherein the compound is a compound of the following formula: 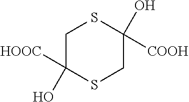
or a pharmaceutically acceptable salt thereof.

12. The method of claim 11 wherein the compound is a pharmaceutically acceptable salt that comprises one or more sodium, choline, or betaine cations.

13. A pharmaceutical composition comprising a compound as described in claim 8, and a pharmaceutically acceptable carrier.

14. A pharmaceutical composition as claimed in claim 13, which is adapted for oral administration.

15. A kit for use in the treatment of a subject in need of treatment for cyanide poisoning, which comprises a compound capable of releasing 3-mercaptopyruvate in vivo, together with instructions for administration of said compound.

16. A kit as claimed in claim 15, which comprises a compound capable of releasing 3-mercaptopyruvate slowly and a compound capable of releasing 3-mercaptopyruvate rapidly.
